(19) United States
(12) Patent Application Publication
(10) Pub. No.: US 2010/0260823 A1
(43) Pub. Date: Oct. 14, 2010

(54) PREPARATION WITH MARINE COLLAGEN FOR PROTEASE INHIBITION

(75) Inventors: Corneliu Iulian Alupei, Neuwied (DE); Peter Ruth, Melsbach (DE); Tim Axel Wilhelms, Koblenz (DE); Christian Rohrer, Linz (AT)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: LOHMANN & RAUSCHER GMBH & CO KG., Neuweid (DE)

(21) Appl. No.: 12/377,407

(22) PCT Filed: Jul. 28, 2007
(86) PCT No.: PCT/EP2007/006694

§ 371 Date: Feb. 13, 2009

(30) Foreign Application Priority Data
Aug. 16, 2006 (DE) 10 2006 038 252.8

(51) Int. Cl. A61F 13/00; A61K 33/40; A61K 38/39; A61P 29/00; A61P 19/02; A61P 35/00; A61P 17/02

(52) U.S. Cl. 424/445; 424/616; 514/17.2

(57) ABSTRACT

The invention relates to pharmaceutical preparations made from marine collagens for inhibiting matrix metalloproteases and the use of a marine collagen for production of a pharmaceutical preparation for inhibiting matrix metalloproteases.

### CROSS-REFERENCE TO RELATED APPLICATIONS

This application is a Section 371 of International Application No. PCT/EP2007/006694, filed Jul. 28, 2007, which was published in the German language on Feb. 21, 2008, under International Publication No. WO 2008/019756 A1 and the disclosure of which is incorporated herein by reference.

### BACKGROUND OF THE INVENTION

The present invention relates to pharmaceutical preparations and pharmaceutical devices containing marine collagen for inhibiting matrix metalloproteinases, as well as to the use of marine collagen for the production of preparations for the treatment and therapy of diseases that are mediated by matrix metalloproteinases.

The matrix metalloproteinases (matrix metalloproteases/matrix metallopeptidases/MMPs) form an enzyme family that is formed in various cells and which is responsible for the extracellular degradation of macromolecules. The name “matrix metalloproteinases” goes back to the fact that the catalytic activity of the proteases is due to a zinc atom in the catalytic center, and that the structural stability of the protein is ensured by potassium.

MMPs are subdivided into four different classes, with this classification being based, inter alia, on the domain structure and the preferred substrates, so that a distinction is made, for example, between collagenases and gelatinases.

The function of the MMPs is to hydrolyze components of the extracellular matrix, such as collagens, gelatine, nectins, laminins, elastin and core proteins of the proteoglycans. Hence, they play a decisive role in the physiological processes of tissue transformation, for example in embryogenesis, growth, but also in wound healing and in pathobiological processes.

Whereas, under “normal conditions,” MMPs can practically not be detected in the skin, tissue and joints, or only in very low concentrations, they appear in increased numbers in the case of injuries to the skin and in diseases such as rheumatism and arthritis. In addition, their effect in tumor formation and metastasis of different tumors could be demonstrated. Thus, MMP-13, for example, is up-regulated in numerous malignant tumors.

Apart from the treatment of the above-mentioned MMP-mediated diseases, such as rheumatism and arthritis, which occur in a plurality of older and increasingly younger patients, the healing of wounds—in which, naturally, elevated MMP-13 concentrations can be detected—has likewise been a problem ever since. This applies particularly to chronic wounds—the term chronic wounds referring to those showing no tendency to heal after 6-8 weeks—from which according to current estimates about four million people are suffering in Germany, and with treatment costs from two to three billion Euros being incurred each year.

As already mentioned, an important factor in the formation of these chronic wounds is the existence of a strong imbalance between the protease concentration (MMP concentration) in the exudation of a wound and the concentration of the corresponding protease inhibitors (TIMP concentration). Normally, the MMPs, which are initially activated after the secretion, are regulated by the TIMPs (tissue inhibitors of metalloproteinases). However, if this delicate balance is disturbed, there is an excess of proteases that not only immediately breaks down newly formed tissue which is required for wound healing and for closure of the wound, but also attacks and lyses healthy tissue and deactivates growth factors, which results in a continuous, sometimes slowly progressing, deterioration of the wound.

On the other hand, studies on wound healing have shown that in MMP-13-deficient wounds wound healing is promoted and accelerated to a great extent.

A greater inhibition and lower activity, or concentration, of MMPs, in particular of MMP-13, would therefore constitute a suitable starting point for the treatment of chronic wounds as well as of arthritic and rheumatic diseases.

In vitro, MMPs can be inhibited by chelating agents, such as EDTA, which bind or block the zinc of the catalytic center or the structurally significant potassium.

Furthermore, numerous pharmacological preparations are known which inhibit the activity of the MMPs. However, these inhibitors often have a low specificity, with the consequence that they also affect other enzymes in addition to the MMPs, or that they even have a cytotoxic or toxic effect on the entire organism.

An alternative treatment method known in the state of the art is, for example, the application of collagen-containing wound dressings, by means of which an alternative substrate is offered to the proteases secreted in the wound, the purpose of this being to inhibit the degrading enzymes competitively.

A problem of this treatment is that the proteases have insufficient specificity towards the collagen dressings used, and that the activity of the MMPs is insufficiently inhibited by the excess of collagen. Another problem is that these collagen dressings are mostly derived from bovine tendons or skins. With this collagen source there exists a latent risk that the wound dressings might contain pathogens causing BSE (BSE=bovine spongiform encephalopathy), which is still a wide-spread fear among consumers. Although by choosing alternative sources, such as horses or pigs, it is possible to circumvent this problem to a large degree, there is still an element of risk of becoming infected with pathogens causing TSE (TSE=transmissible spongiform encephalopathy).

### BRIEF SUMMARY OF THE INVENTION

It was thus the object of the present invention to provide a pharmaceutical preparation or a pharmaceutical device which inhibits the activity of the MMPs and improves wound healing and chronic-inflammatory diseases without presenting the inherent risk of an infection with pathogens causing spongiform encephalopathy and without showing toxic effects or having any other adverse drug effects.

It has, surprisingly, been found that pharmaceutical preparations containing collagen that has been obtained from marine organisms (marine collagen), especially collagen obtained from Chondrosia reniformis, have a strongly inhibiting effect on MMP activity and particularly on MMP-13, with the consequence that it is possible to use pharmaceutical preparations based on that collagen for treating MMP-mediated diseases. A comparison with commercial collagen preparations (e.g. made of bovine or porcine collagen) showed no impairment of the activity of the proteases compared to the control experiment. Marine collagen, as according to the present invention, is here defined as collagen that is obtained from marine sponges (Porifera) or other marine organisms that have no nervous system.

Another advantage of these preparations containing marine collagen is that the source organism does not have a nervous system, so that the risk of a transmission of pathogens causing TSE and of a concomitant infection is entirely impossible.

Furthermore, the inventive preparations possess only a very low allergenic potential, and the preparations can be completely catabolized by the organism.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a bar graph plotting MMP-13 concentration against time to illustrate inhibition of MMP-13 activity by various collagen sponges compared to a control, as described in Example 6.

### DETAILED DESCRIPTION OF THE INVENTION

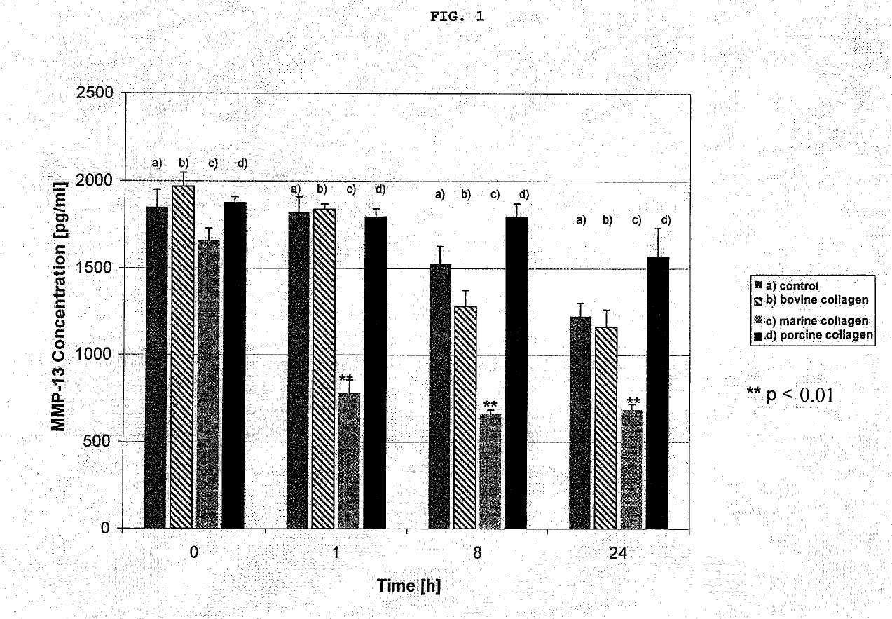

As already explained, preparations containing treated marine collagen, preferably obtained from sponges and more preferably from Chondrosia reniformis, significantly inhibit the activity of MMPs (see FIG. 1). The decrease in the concentration, and thereby in the activity, of the MMPs becomes apparent already one hour after application of the preparation. It is not necessary to add further inhibitors since, in contrast to the known preparations, the collagen preparations of the present invention sufficiently inhibit and bind MMPs.

The collagen used for the pharmaceutical preparations of the present invention is obtained by extraction of the collagen fraction of Chondrosia reniformis and subsequent purification of the extract, thereby obtaining a collagen solution.

In one embodiment the pharmaceutical preparation is present as a sterile collagen solution. This solution can be used both for the treatment of wounds and for intracorporal application, for example for injections into the joint in cases of arthropathy, such as arthrosis.

For wound treatment, commercial, collagen-free wound dressings, for example, can be soaked in the inventive collagen solution.

According to another embodiment, the collagen is precipitated from the prepared solution by changing the pH value or by adding ethanol or other, suitable, physiologically acceptable solvents so as to produce a colloid or a dispersion.

The viscosity of the colloid or dispersion thus obtained can be set by reducing the solvents, for example by withdrawing the solvents under vacuum or by centrifuging, or by viscosity modifiers, such as PVP (polyvinyl pyrrolidone), polyacrylates or cellulose derivatives, for example carboxymethyl cellulose (CMC), hydroxyethyl cellulose (HEC), hydroxyethyl propyl cellulose (HEPC) or methyl cellulose (MC), so that a gel or a highly viscous solution is obtained.

Both the dispersion or colloid, and the gel can then be used for treating wounds or as injections into a joint for the treatment of arthrosis.

According to a further embodiment, the purified collagen is precipitated from the solution, dried, and processed into granules that are incorporated in preparations, such as creams and ointments or wound dressings.

In a preferred embodiment, the inventive preparations are porous sponges containing a marine collagen, preferably a collagen obtained from Chondrosia reniformis and that has been treated according to the process described in Example 1, with wound dressings for MMP inhibition which accelerate the closure of wounds, more preferably consisting exclusively of marine collagen.

The collagen preparations of the present invention may, in addition to other active agents and pharmaceutical additives, contain at least one further active agent that does not inhibit the MMPs, for example non-steroidal antirheumatics, such as acetylsalicylic acid or ibuprofen, and/or antibiotics.

To produce the wound dressings, a collagen solution that has been extracted from Chondrosia reniformis and treated in accordance with the prescription provided in German published patent application DE 10 2005 008 416 A1 (see Example 1) is freeze-dried, with a porous collagen sponge being obtained thereby. The collagen sponge thus obtained may be divided into flat pieces of appropriate size and stored in a sterile package until it is used. To improve the storage life of the product, and to prevent an infection by any pathogens that have remained in the product, antimicrobial active agents may be added to the collagen solution, the packaged units may be subjected to irradiation (dose: 25 kGy) and/or they may be gassed with ethylene oxide.

The advantages of the wound dressings thus obtained consist in that they do not present any risk whatsoever of causing an infection with TSE pathogens, and that the MMPs present in the wound exudate, especially MMP-13, are deactivated and the wound healing is promoted. The deactivation of the MMPs here is due, on the one hand, to a competitive inhibition of the substrate, since either it is the marine collagen present in the wound dressings that is degraded primarily, or, by contrast, the degradation of the marine collagen takes place more slowly than that of the natural substrate. On the other hand, it is due to the fact that exudate absorbed into the dressing is bound and the MMP-13 is immobilized, with the consequence that a “reflux” to the wound surface, and thereby the degradation of newly formed structures, is prevented and wound healing is improved.

As can be seen from FIG. 1, a marked reduction in MMP-13 activity is observed already 60 min after addition of the collagen, whereas with commercial collagen preparations a considerably higher activity of MMP-13 and no change in comparison to the control experiment is observed. However, as already described above, the rapid and efficient reduction in MMP-13 activity, as shown in FIG. 1, is one of the basic prerequisites for inducing the wound healing process in chronic wounds. With the preparations according to the invention, addition of another MMP-13 inhibitor, for example an unspecific chelating agent, is not necessary.

The immobilization of the MMPs in the collagen matrix can, in accordance with another embodiment, be increased by a semi-permeable membrane being arranged on the collagen sponge on the side facing the wound, said semi-permeable membrane permitting the absorption of exudate into the sponge but preventing the reflux of exudate and of degradation products.

An additional advantage of the wound dressings thus prepared is their low allergenic potential, which is due to their high purity, and their being completely biologically degradable, even in the wounds.

Apart from collagen sponges, it is also possible to produce collagen films if modified methods are employed, such as spread-coating and drying.

In addition, it is possible to coat suitable carrier materials, such as films and textile fabrics, with collagen films and collagen sponges, wherein said films and fabrics may form an impermeable backing layer or a semi-permeable backing layer.

According to a further embodiment, the backing layer is applied by way of addition to one of the above-described dressings, with the surface of the backing layer preferably being larger than the surface of the collagen sponge and the projecting areas being coated with an adhesive which does not irritate the skin, so that the wound dressing can be fixed on the skin, above the wound.

The above-mentioned inventive pharmaceutical preparations and devices may furthermore contain preservatives and antimicrobial active compounds (e.g., silver sulfadiazine, biguanides, polyhexanide, nitroxoline, octenidine, taurolidine, chlorhexidine, benzalkonium halogenides and pharmacologically acceptable salts or derivatives of the aforementioned compounds), viscosity modifiers (e.g., polyvinyl pyrrolidone or acrylates), growth factors and other wound healing factors, skin protection agents (fatty acids, fatty acid esters) and the like.

Moreover, an additional sterilization of the products by means of irradiation (25 kGy) or by means of gassing with ethylene oxide or with other suitable agents known to those skilled in the art is provided for.

Preferred embodiments of the invention will be described in the following specific, non-limiting examples.

### EXAMPLE 1

### Treatment of Collagen

In accordance with the method described in DE 10 2005 008 416 A1, a collagen precipitate from Chondrosia reniformis, precipitated in an acid medium (pH 3), was separated from the medium by filtration, and the moisture was reduced to a residual moisture content of around 84 wt %. Then, 121 grams of the collagen raw mass was suspended in 1300 ml of an aqueous 0.5% (vol/vol) H₂O₂ solution while stirring for two hours, and the pH of the solution is adjusted to a value of 12.4 with a 5 N NaOH solution in order to dissolve the collagen fibers. The resultant collagen solution was filtered in order to remove non-dissolvable constituents and was subsequently added, under vigorous stirring, to 2600 ml ethanol (conc. 98%) or, deviating from DE 10 2005 008 416 A1, to an HCl solution with a pH of 0-3 and during this addition was kept within the limits of 0-3, which resulted in the precipitation of the collagen in fibrous form in a white or slightly yellowish color. The collagen fibers were separated from the medium by filtration, then freed from adhering moisture and subsequently suspended homogenously, under stirring, in 300 ml of ultrapure water. The pH of the suspension was adjusted with a 5 N HCl solution to a value of 6.5. The collagen solution thus obtained had a concentration of collagen of 2.8 wt %. All process steps were carried out at room temperature.

To obtain a sterile collagen solution according to an alternative embodiment, all of the objects coming into contact with collagen were rinsed with a 0.5% (vol/vol) H₂O₂ solution before they were used, and the precipitated collagen fibers were not suspended in water but in 300 ml of an aqueous 0.5% (vol/vol) H₂O₂ solution.

### EXAMPLE 2

From the preparation prepared in Example 1, there is prepared a cream or ointment for cutaneous application, using thickening agents (e.g., PVP (polyvinyl pyrrolidone), polyacrylates or other cream bases and ointment bases known to those skilled in the art), said cream or ointment, upon application in a wound, inhibiting the MMPs and promoting the closure of the wound.

A particular advantage of this composition is the hydrogen peroxide fraction of the collagen solution remaining in the ointment, which fraction on the one hand increases the storage stability of the preparation and, on the other hand, develops antiseptic action in the wound.

In another embodiment, the collagen solution may be heated shortly or a reducing agent or catalyst may be added thereto in order to destroy the residual peroxide.

### EXAMPLE 3

The collagen solution prepared according to Example 1 is adjusted to a collagen concentration of 1-2% using 0.5% hydrogen peroxide solution or ultrapure water. In the preferred embodiment, the collagen concentration is adjusted to 1 wt % and the pH value is adjusted to 6.1. The solution is placed in a dish and freeze-dried. The collagen sponge thus contained can be used as a wound dressing for treating poorly healing or chronic wounds.

### EXAMPLE 4

In another embodiment, antibiotic or antimicrobial active agents are added to the collagen solution prior to lyophilization.

To this end, an antimicrobial substance, which has previously been dissolved in 0.5% (vol/vol) H₂O₂ solution or in ultrapure water, is added, under rigorous stirring, to the collagen solution prepared according to Example 1, until the collagen content is 1 wt %. The concentration of the antimicrobial substance is between 0.5-2 wt %, relative to the dry weight of the collagen.

Preferably, the antimicrobial substance is polyhexamethylene biguanide hydrochloride (degree of polymerization=12-18) with a concentration of 1 wt %, relative to the dry weight of the collagen. The antimicrobial solutions or dispersions thus prepared are placed in a dish and frozen, and are subsequently freeze-dried.

### EXAMPLE 5

In a further embodiment example, an antimicrobial wound dressing is prepared by impregnation with an antimicrobial substance.

To this end, collagen sponges prepared according to Example 3 are impregnated with an antimicrobially active substance, using methods known in the state of the art (e.g., spread-coating or spraying). At this, a concentrated aqueous or alcoholic solution (10-50%) of an antimicrobial substance is prepared and, by spraying or coating, is applied to a collagen sponge, so that a concentration of the antimicrobial substance of 0.5-2 wt %, relative to the dry weight of the collagen, is obtained.

In one preferred embodiment, an aqueous or an ethanolic polyhexamethylene biguanide hydrochloride solution (20 wt %) is applied, by spraying, onto the collagen foam at a concentration of 50 μl solution per gram of collagen foam, so that a polyhexamethylene biguanide hydrochloride concentration of 1 wt %, relative to the dry weight of the collagen, is obtained.

### EXAMPLE 6

The experiments for determination of the MMP-13 inhibition by the inventive collagen preparations were carried out as follows:

1. Sample Preparation

For the experiments on MMP-13 inhibition, a solution with a concentration of 2000 pg/ml was prepared from lyophilized protein standards.

Pieces of a uniform size (0.5 cm² each) were punched out of the preparations prepared according to the present invention as well as from commercial collagen sponges, using an 8-mm-biopsy punch (Stiefel Laboratorium GmbH, Offenbach, Germany), and these were transferred to a 24-well cell culture plate. Each sample was taken up in 1 ml of protein solution and was subsequently incubated for 24 hours on an agitator (ThermoStar™, BMG Labtech GmbH, Offenburg, Germany) at 37° C. To monitor the MMP-13 concentration, the supernatant was removed after 0, 1, 8 and 24 hours, and the samples were frozen immediately at −20° C. until the measurement was carried out. As a control experiment, samples containing no collagen preparation were measured.

2. Determination of MMP-13 Concentration

The matrix metalloproteinase-13 6concentrations were quantified using enzyme-linked immunosorbent assays (Quantikine™ pro-MMP-13 Immunoassay DM1300, R&D Systems GmbH, Wiesbaden, Germany). The determination of the MMP concentration was carried out in the plate reader (Fluostar™, BMG Labtech GmbH, Offenburg, Germany) by measuring the optical density (OD) at 450 nm (reference wavelength: 620 nm). Subsequently, the enzyme concentration could be calculated on the basis of a “lin-log” plot (OD or fluorescence—linear scale; concentration—logarithmic scale) by means of a 4-parameter fit. All samples were measured by repeat determination, the values obtained were averaged and the standard error was calculated.

For each time of measurement, the MMP-13 concentration was determined by two to four independent assays. The individual measurements were performed by repeat determination. The concentrations indicated in FIG. 1 are the averaged values from 4, 6 and 8 measured data, respectively, taking into account the standard errors. To determine the statistical significance, a simple analysis of variance (ANOVA one way) was made.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1-18. (canceled)

19. A method for treating a matrix metalloproteinase-mediated disease selected from rheumatism and arthritis, the method comprising administering a pharmaceutical preparation containing a marine collagen originating from sponges and an antibiotic, the preparation being in a form selected from a solution, a suspension, a dispersion, a colloid, a gel, a cream or an ointment.

20. The method for treating a matrix metalloproteinase-mediated disease selected from malignant neoplasms, the method comprising administering a pharmaceutical preparation containing a marine collagen originating from sponges.

21. The method for treating a matrix metalloproteinase-mediated disease selected from wounds, the method comprising administering a pharmaceutical preparation containing a marine collagen originating from sponges and an antibiotic, the preparation being present in a form selected from a solution, a suspension, a dispersion, a colloid, a gel, a cream, an ointment, a film, or a wound dressing comprising a backing layer made of film or of textile fabric, wherein the backing layer is coated with a sponge made of the marine collagen.

22. The method according to any claim 19, wherein the marine collagen originates from the sponge Chondrosia reniformis.

23. The method according to claim 19, wherein the preparation further contains components and pharmaceutical additives in addition to the marine collagen.

24. The method according to claim 19, wherein the pharmaceutical preparation contains preservatives and/or antimicrobially active compounds selected from silver sulfadiazine, biguanide, polyhexamethylene biguanidine, polyhexamethylene biguanidine hydrochloride, nitroxoline, octenidine, taurolidine, chlorhexidine and benzalkonium halogenide and/or pharmacologically acceptable salts or derivatives of these compounds.

25. The method according to claim 19, wherein the pharmaceutical preparation further contains at least one additional active agent which does not inhibit matrix metalloproteinases and which is selected from the group of non-steroidal antirheumatics.

26. The method according to claim 25, wherein the non-steroidal antirheumatic is acetylsalicylic acid or ibuprofen.

27. The method according to claim 21, wherein the backing layer of the wound dressing is impermeable or semi-permeable.

28. The method according to claim 21, wherein a surface area of the backing layer is larger than a surface area of the sponge.

29. The method according to claim 28, wherein projecting areas of the backing layer are coated with a pressure-sensitive adhesive tolerated by the skin.

30. The method according to claim 19, wherein the preparation was sterilized by gassing with ethylene oxide or by irradiation with 25 kGy.

31. A pharmaceutical preparation for treating a matrix metalloproteinase-mediated disease selected from rheumatism and arthritis, wherein the preparation contains marine collagen originating from sponges and contains an antibiotic and is present in a form of a solution, a dispersion, a colloid or a gel.

32. A pharmaceutical preparation for treating a matrix metalloproteinase-mediated disease selected from malignant neoplasms, wherein the preparation contains marine collagen originating from sponges and is present in a form of a solution, a dispersion, a colloid or a gel.

33. A pharmaceutical preparation for treatment of wounds, wherein the preparation comprises marine collagen originating from sponges and contains an antibiotic and is present in a form of a solution, a suspension, a dispersion, a colloid, a gel, a cream, an ointment, a film, or a wound dressing comprising a backing layer made of film or of textile fabric, wherein the backing layer is coated with a sponge made of the marine collagen.

34. The pharmaceutical preparation according to claim 32, wherein the marine collagen originates from the sponge Chondrosia reniformis.
